(19) United States
(12) Patent Application Publication
(10) Pub. No.: US 2020/0390429 A1
(43) Pub. Date: Dec. 17, 2020

(54) THROUGH-THE-SHEATH TRANS-RADIAL CLOSURE DEVICE, DEPLOYMENT APPARATUS, AND METHOD OF DEPLOYMENT

(71) Applicant: Transluminal Technologies, LLC, Syracuse, NY (US)

(72) Inventors: Stephen M. Green, Syracuse, NY (US); Ronald P. Caputo, Manlius, NY (US); John M. Kirwan, Wilbraham, MA (US)

(73) Assignee: Transluminal Technologies, LLC, Syracuse, NY (US)

(21) Appl. No.: 16/904,084

(22) Filed: Jun. 17, 2020

Related U.S. Application Data
(60) Provisional application No. 62/862,151, filed on Jun. 17, 2019.

(51) Int. Cl. A61B 17/00; A61L 17/04; A61L 31/02

(52) U.S. Cl. CPC A61B 17/0057; A61L 17/04; A61L 31/022; A61B 2017/00778; A61B 2017/00004; A61B 2017/00659; A61B 2017/00663

(57) ABSTRACT

The present invention provides an apparatus and method for creating hemostasis at a subcutaneous vascular puncture. The method and apparatus is intended, but not limited to, vascular punctures following trans-radial arterial procedures, e.g. catheterization and percutaneous coronary intervention.

### CROSS-REFERENCE TO RELATED APPLICATIONS

The present application claims priority to and the benefit of U.S. Provisional Patent Application No. 62/862,151 filed on Jun. 17, 2019.

### FIELD OF THE INVENTION

The present invention relates to medical devices, and more particularly, to a vascular puncture hemostasis apparatus following trans-radial arterial procedures.

### BACKGROUND OF THE INVENTION

Various medical procedures, particularly cardiology procedures, involve accessing a corporeal blood vessel through a percutaneous sheath. Insertion of the sheath necessarily requires an opening, or puncture wound, in the blood vessel so that a medical procedure can be performed through the sheath. After the medical procedure has been completed, the sheath must be removed from the blood vessel and the access hole in the blood vessel must be closed to create cessation of bleeding from the blood vessel.

As an alternative to the historically standard access to the cardiovasculature via the femoral artery in a patient's groin, access via an artery in a patient's wrist (i.e. either the radial artery or the ulnar artery) has gained recent popularity. This is particularly due to lessened post-procedure access site bleeding complications. The standard means for inducing post-procedure hemostasis of either a radial artery or an ulnar artery is to apply direct pressure to the patient's wrist approximate of the subcutaneous sheath entry site, or arteriotomy. Several devices have been introduced into the device market which aid in applying such direct pressure to a patient's wrist. These hemostasis devices are frequently composed of a wrist band with a means for focusing direct contact pressure on the patient's inside wrist skin surface approximate to the subcutaneous vessel's puncture wound. Such wrist band type devices may incorporate an inflatable balloon element for further focusing the direct pressure at the specific position on the patient's wrist.

A complication that can arise from compression type hemostasis devices is for the artery to collapse and become occluded, owing to the applied direct contact pressure. Such collapsing of a radial artery for instance, and the resulting non-patency, can create reduced blood flow to the patient's hand, as well as render the radial artery unusable for future percutaneous procedures. Arterial occlusion occurs in approximately 5-12% (see e.g. Agostoni P, Biondi-Zoccai G G, de Benedictis M L, et al. Radial versus femoral approach for percutaneous coronary diagnostic and interventional procedures. Systematic overview and meta-analysis of randomized trials. J Am Coll Cardiol. 2004; 44:349-356; Bertrand O F, Rao S V, Pancholy S, et al. Transradial approach for coronary angiography and interventions: results of the first international transradial practice survey. J Am Coll Cardiol Interv. 2010; 3:1022-1031; Rashid M, Kwok C S, Pancholy S, Chugh s, Kedev S A, Bernat I, Ratib K, Large A, Fraser D, Nolan J, Mamas M A. Radial artery occlusion after transradial intervntions: A systemic overview and meta-analysis. J Am Heart Assoc. 2016; 5:e002686 doi: 10.1161/JAHA.115.002686.) of patients undergoing procedures through the radial artery approach and therefore relates to a substantial patient population, particularly in high volume hospitals.

The present invention offers a means for facilitating hemostasis at a radial or ulnar artery puncture while avoiding the deleterious conditions that can result from arterial occlusion after administering direct pressure at the access site.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### SUMMARY OF THE INVENTION

It is therefore a principal object and advantage of the present invention to provide a tissue puncture closure assembly comprised of a closure device for insertion into and sealing of a blood vessel wall puncture that overcomes the shortcomings of direct overlying compression type closure systems described supra.

Another object and advantage of the present invention is the use of expansive force rather than compressive force against the wall of a blood vessel, i.e. force applied to the inner aspect of the artery to achieve arteriotomy closure.

It is a further an object and advantage of the present invention to provide a closure implant that rapidly and completely dissolves (biodegrades) in vivo, allowing for future arterial access, i.e. ‘re-sticks’.

It is a further object and advantage of the present invention to provide a closure device that can be used through the existing procedural sheath avoiding the necessity to withdraw the sheath and introduce a closure device over a guidewire. In accordance with the foregoing object and advantages, an embodiment of the present invention provides a closure device that includes an absorbable anchor, or footplate, for insertion through the blood vessel wall puncture.

Embodiments of the footplate comprise a biocompatible and biocorrodible metal comprising a magnesium alloy, e.g. Mg1Al, Mg3Al, Mg6Al, Mg8Al, Mg10Al, Mg12Al. Dissolution, or absorption, of magnesium alloy in vivo is an electrochemical corrosion process whereby blood, or bodily fluid, acts as an electrolyte and the Mg alloy implant, with its greatly negative electrochemical potential, acts as a ‘sacrificial anode’ in the resulting electrochemical cell formed at the implant's surface. As such, the dissolution of an absorbable magnesium alloy implant is a surface phenomenon. Therefore, the time to complete absorption of the footplate can be altered by adjusting the surface area-to-volume ratio. Further, the chemical constituency of the alloy can greatly influence the rate at which the implant, or footplate, dissolves, or absorbs. The footplate embodiments presented herein are configured to have a maximized surface area-to-volume ratio, and further are comprised to have a chemical constituency such that when presented inside the blood vessel, will completely dissolve in a period of hours, or preferably, shortly after the procedure and before the patient leaves the hospital.

The rate of dissolution of the magnesium alloy footplate can be further accelerated by surface modification, i.e. surface pretreatment. In brief, the process involves contacting the alloy with a specially prepared aqueous solution by dipping, spraying, or brushing followed by rinsing and drying in clean water. The solution is defined by the addition of a suitable acid to activate the alloy and modify the pH of the solution, and an accelerant, which is specifically selected to achieve increased corrosion. Through this process, the surface composition of the alloy is modified by (1) enriching it in impurities already contained in the alloy as the Mg component corrodes preferentially, and (2) depositing product(s) from solution that are associated with the acid and accelerant addition. Suitable inorganic acids include sulfuric, nitric, hydrochloric, and phosphoric and phosphonic. Acid concentrations may range from 1 mg to 10 g per liter of solution. Suitable organic acids include citric, tartaric, acetic, and oxalic. Suitable accelerants are generally soluble transition metal salts, typically though not exclusively of iron, manganese, and cobalt. Accelerant concentrations are typically much less than acid concentrations and range from 0.01 to 1 g per liter of solution. The contact time between solution and treated surface may be varied to further adjust corrosion rate. Contact times may range from 5 seconds to 10 minutes based on the chemistry of the pretreatment solution and the alloy. After pretreatment, surfaces are rinsed thoroughly with distilled or deionized water to halt the interaction between the pretreatment solution and the alloy. No further treatment of the surface is needed prior to use. An example of the process is as follows.

Surface Pretreatment Example

Mg alloy samples are pretreated by immersion in an aqueous solution of 1 g of 98% sulfuric acid H₂SO₄ and 0.04 g of ferrous sulfate FeSO₄ in 10 mL of distilled water. Samples are treated in batches of 25 for a minimum of 90 seconds and no longer than 120 seconds. Samples are rinsed and dried in air after immersion in the pretreatment solution. At this stage the samples are complete and ready for use.

Apart from the benefits of rapid dissolution, magnesium implants have the added attribute of possessing antibacterial properties, i.e. properties that prevent implant-associated infection. In various studies (see e.g. Rahim M I, Eifler R, Rais B, Mueller P P, Alkalization is responsible for antimicrobial effect of corroding magnesium. Journal of Biomedical Materials Research. 2015: 103, 11: 3526-3532, doi: 10.1002/jbm.a.35503; Yang L, Guangwang L, Zanjing Z, Lina L, Haowei L, et al. Antibacterial properties of magnesium in vitro and in an in vivo model of implant-associated methicillin-resistant Staphylococcus aureus infection. Antimicrobial Agents and Chemotherapy. 2014: 58, 12: 7586-759, 1doi: 10.1128/AAC.03936-14; Robinson D A, Griffith R W, Scechtman D, Evans R B, Conzemius M G, In vitro antibacterial properties of magnesium metal against Escherichia coli, Pseeudomonas aeruginosa and Staphylococcus aureus. Acta Biomaterialia. 2010: 6: 1869-1877, doi: 10.1016/j.actbio.2009.10.007) the proliferation of bacteria (e.g. Escherichia coli, Pseudomonas aeruginosa, Staphylococcus aureus) has been shown to be suppressed in the presence of metallic magnesium, similarly to the effects of bacterial antibiotics. These studies indicate that the antibacterial activity associated with metallic magnesium is largely attributable to an increase in pH (i.e. increase in alkalinity) at the implant site.

Embodiments of the footplate can be fabricated by using a number of manufacturing techniques. These include, but are not limited to, molding, extruding, machining, stamping, casting, forging, laser cutting and/or processing, laminating, adhesively fixing, welding, combinations thereof, among others, with effectiveness, as needed or desired.

Embodiments of the filament may include either a bioabsorbable or non-bioabsorbable material, both types of which are commercially available for use as sutures. Bioabsorbable filaments may be comprised of various hydrolysis-dissolvable materials including Polyglycolic Acid polymer, Polyglactin copolymer, Poliglecaprone copolymer, Polydioxanone polymer, or Catgut. Non-bioabsorbable filaments may be comprised of such materials as Polypropylene, Nylon (polyamide), Polyester, PVDF, PTFE, ePTFE, silk, stainless steel, or nitinol. Further, embodiments of the filament may be of either a braided or monofilament construction.

In accordance with an embodiment of the present invention, the absorbable magnesium alloy footplate includes a single thru-hole which provides passage of a single non-absorbable filament. At its most distal end (on the lumen side of the footplate) the filament is locally compressed, or flattened, as to create a geometry that is sufficiently larger than the diameter of the thru-hole in the footplate, thus providing a stop such that the footplate remains attached, or tethered, to the filament. At such time that the footplate has sufficiently dissolved inside the blood vessel, the filament is completely removed from the patient by applying a pulling force to the end of the filament that extends outside of the patient's body.

Another embodiment of the present invention provides an absorbable magnesium alloy footplate with a single pierced or punched thru-hole, or fenestration, through which an absorbable or non-absorbable filament either partially or fully extends. The fenestration can be provided at a variety of angles relative to the flat surface of the footplate, e.g. perpendicular or at acute angles. Further, the geometry of the mandrel that pierces the fenestration can be configured in a variety of geometries (e.g. round, oval, square, rectangular) and with various tip geometries (e.g. pointed, sharpened, blunt) as to create a trailing fenestration geometry through which the filament can pass. The fenestration can then be reformed back to a substantially flat geometry (planar with the surface of the footplate) such that the filament is securely captured, owing to the deformability of the relatively soft magnesium alloy. In the embodiment that includes a non-absorbable filament, at such time that the footplate has sufficiently dissolved, thereby providing mechanical detachment of the filament from the footplate, the filament is completely removed from the footplate and the patient by applying a pulling force to the end of the filament that extends outside of the patient's body. In the embodiment that includes an absorbable filament, at such time that the footplate has sufficiently dissolved, the absorbable filament is cut just below the level of the skin and the remaining filament bioabsorbs in the subcutaneous tissue underlying the skin.

The invention further provides a delivery assembly for presenting the footplate and filament into the blood vessel through a pre-existing, in-place, introducer sheath and articulating the footplate to a position substantially parallel to the longitudinal axis of the blood vessel. The delivery assembly is comprised of two concentric cannulae (a delivery shaft and a pusher tube) with the footplate nested in the distal margin of the delivery shaft and the filament extending proximally and internal to the pusher tube. In a single ‘push cycle’, the concentric cannulae act together to slide-ably deliver the footplate to the inside of the blood vessel, i.e. the internal pusher tube applies a distally directed force to the footplate, thus motivating it in a distal direction relative to the delivery shaft resulting in the footplate being extruded out of the delivery shaft and into the blood vessel.

Embodiments of the outer cannula, or delivery shaft, may be comprised of biocompatible, appropriately flexible, kink resistant, low durometer polymers such as Pebax, Silicone, Nylon (polyamide), Polyurethane, PTFE, FEP, ETFE, HDPE, etc. Delivery shaft embodiments can be fabricated by using a number of manufacturing techniques. Those include, but are not limited to, molding, extruding, machining, laser cutting and/or processing, radio frequency (RF) forming and/or tipping, adhesively fixing, with effectiveness, as needed or desired.

Embodiments of the inner cannula, or pusher tube, may be comprised of biocompatible, appropriately stiff, kink resistant, polymers such as Pebax, Silicone, Nylon (polyamide), Polyurethane, PTFE, FEP, ETFE, HDPE, etc., or alternatively, may be comprised of a stainless steel hypodermic tubing. Pusher tube embodiments can be fabricated by using a number of manufacturing techniques. Those include, but are not limited to, molding, extruding, machining, laser cutting and/or processing, radio frequency (RF) forming and/or tipping, adhesively fixing, with effectiveness, as needed or desired.

In an embodiment of the present invention, the outer cannula, or delivery shaft, houses the pusher tube, footplate, and filament. The inside diameter of the delivery shaft is configured to be larger than the outside diameter of the pusher tube. In the default position, the pusher tube's distal tip is in contact with the proximal end of the footplate. The distal end of the delivery shaft is configured with two axial slits positioned 180 degrees from one another such that the footplate is nested and held in frictional engagement with the slits.

In another embodiment, the delivery shaft may include a closed or semi-closed distal end that provides an atraumatic geometry (spherically or semi-spherically configured) inside of which the footplate resides in the default position. The slit distal margin of the delivery shaft extends through the closed distal tip thus providing an atraumatic and protective covering over the distal margin of the footplate.

Another aspect of the present invention provides an axial displacement of the pusher tube which is substantially greater than the length of the footplate such that when the pusher tube is motivated (pushed) in the distal direction, the footplate is displaced (extruded) from within the delivery shaft to a position well distal of the distal end of the delivery shaft. Further, because the delivery shaft's inside diameter is smaller than the width of the footplate; the footplate is prevented from re-entering the delivery shaft. When fully displaced in the distal direction relative to the delivery shaft, the footplate articulates to an orientation substantially perpendicular to the longitudinal axis of the delivery sheath and thereby substantially parallel with the longitudinal axis of the blood vessel. This articulation results from the elastic straightening of the filament when the footplate and the filament are released from their nested position within the delivery shaft, i.e. when the distal portion of the filament is freed from having a 90 degree bend to being substantially straight. The resulting articulation, or rotation, of the footplate aids in disallowing the footplate from inadvertently exiting the blood vessel once introduced and then, when the delivery assembly is subsequently retracted proximally, the footplate is positioned to reliably approximate against the wall of the blood vessel to effect hemostasis.

The closure device is intended to be used in combination with an introducer sheath which is the essential conduit by which access to the cardiovasculature is achieved. At the end of the interventional procedure, the introducer sheath is left in place (in the patient's wrist) and the closure device is inserted completely through the introducer sheath and mated to the proximal end of the introducer sheath, thus forming a combined closure device/introducer sheath assembly. After insertion of the closure device into and through the introducer sheath, the footplate and filament are deployed inside the lumen of the blood vessel to facilitate hemostasis.

Another aspect of the present invention utilizes a radiopaque marking, or marker band, that is often integrated at or near the distal margin of commercially available introducer sheaths. Such marker bands allow the operator to visualize the position of the introducer sheath with respect to the vessel lumen and the vessel wall with the aid of fluoroscopy. Further, as utilized with the closure device, such a radiopaque 360 degree marker band provides the operator with additional feedback related to the proper positioning of the closure device/introducer sheath combined assembly (within the arterial lumen) prior to deployment of the closure device. When fully mated, the position of the closure device/introducer sheath combined assembly may be adjusted (pulled proximally or pushed distally) as a unit such that the distal margin of the combined assembly is at or near the vessel wall. This positioning allows for the closure device to be deployed at a position at or near the vessel wall, thus avoiding inadvertent snagging of the footplate/filament (the implant) at a proximal position in the patient's artery. Such marker bands are typically comprised of a biocompatible, highly loaded tungsten-filled thermoplastic polymer that may be heat fused to the surface of the delivery sheath, or alternatively, a biocompatible ink comprising radiopaque fine particles of materials such as platinum, tungsten, or barium sulfate.

Another aspect of the present invention provides a biocompatible hydrophilic coating that may be applied and bound to the surface of the delivery shaft. Such hydrophilic coatings absorb and bind ie hydrophilic surface, i.e. induce dynamic hydrogen bonding with surrounding water. These chemical interactions with water give rise to hydrogel materials that exhibit extremely low coefficients of friction, thus greatly improving lubricity. As applied to the delivery shaft, once wetted with a sterile saline solution, the friction at the interface between the outside surface of the delivery shaft and the inside surface of the introducer sheath may be greatly reduced as the closure device is inserted distally through the introducer sheath.

Another aspect of the present invention provides a method by which an operator may manipulate the delivery assembly after the footplate has been delivered inside the blood vessel lumen (as described supra). The method provides a deployment technique for seating the footplate, tensioning the filament and securing the filament to provide temporary mechanical contact of the footplate with the blood vessel wall. The method includes the following steps.

Once the footplate has been successfully delivered inside the blood vessel lumen, the closure device/introducer sheath combined assembly is retracted proximally as a single unit such that the footplate is approximated against the blood vessel's inside wall. When the footplate is fully approximated (i.e. engaged with the vessel wall), the combined assembly is pulled further proximally (away from the patient) such that the combined assembly (mated closure device and introducer sheath) fully exits the percutaneous tissue tract. This proximal motion, simultaneously applies continuous tension to the filament. Once the delivery assembly has been pulled proximally to the point where the filament is exposed, proximal of the skin incision (outside of the patient's body), the user grasps the filament and applies gentle tension, which maintains contact force of the footplate against the blood vessel wall, and provides hemostasis at the arteriotomy. Then, further pulling of the delivery assembly completely detaches the delivery assembly from the filament. The filament, having gentle tension applied to it, can be taped to the patient's arm in order to maintain tension on the filament and adequate contact force between the footplate and the artery wall, i.e. to maintain hemostasis.

The footplate is further aided in maintaining contact with the vessel wall by the patient's positive blood pressure that acts on the exposed surface of the footplate. The combination of the filament tethering the footplate and the positive blood pressure is sufficient to resist migration of the footplate during the time it takes for the footplate to dissolve. The filament remains fixed to the patient's anterior wrist skin surface via mechanical securement (e.g. adhesive tape, alligator clip, etc.) for a period of time such that the footplate has substantially dissolved within the artery.

At such time that the footplate has substantially dissolved, or absorbed, the filament can be pulled in a proximal direction (away from the patient) while simultaneously holding pressure on the patient's skin such that the operator's forefinger and middle finger straddle the filament thus providing equal and opposite downward contact force on the skin and subcutaneous tissue while tension is applied to the filament during removal. This technique provides support of the subcutaneous tissue and underlying blood vessel wall, thereby lessening the likelihood that the filament removal will dislodge the thrombus plug at the arteriotomy. At this juncture, the filament has been completely removed from the patient and there exists a fully hemostatic condition at the puncture wound (arteriotomy) due to the natural coagulation of blood and resulting thrombus plug that is formed at the arteriotomy.

As described supra, in the embodiments that include a single, non-absorbable filament extending proximally, the single filament is pulled in a proximal direction in order to remove it from the patient. In the embodiments that include a single absorbable filament, the filament is intended to absorb in the subcutaneous tissue overlying the blood vessel. In such an absorbable filament embodiment, the portion of filament that extends proximally outside of the patient is cut just beneath the patient's skin and then disposed of.

In another embodiment of the present invention, the taping of the filament is replaced with a mechanical cinching device that comes into contact with the patient's skin and possesses adequate clamping force to maintain the necessary tension on the filament. One example of such a cinching device is commonly referred to as a “cord lock”.

In another embodiment of the present invention, the mechanical cinching device may be a smooth jawed ‘alligator clip’, clamped over the filament at the skin surface with adequate clamping force to maintain the necessary tension on the filament and secure the footplate.

In another embodiment of the present invention, the filament may be captured in a slit in a retaining pad as the filament exits the skin incision. The interference between the filament and the slit in the retaining pad provides sufficient tension on the filament to keep the footplate in place. The retaining pad may be comprised of a biocompatible, appropriately dense, open or closed cell rubber such as a closed cell polyurethane foam.

Another embodiment of the present invention provides a hemostatic pad that is incorporated with the filament locking means (e.g. an alligator clip) to aid in inducing hemostasis at the skin incision and the underlying tissue. The hemostatic pad is positioned to be in direct contact with the patient's skin, i.e. between the patient's skin and the filament locking device (e.g. an alligator clip) such that it is trapped to maintain firm contact against the skin. The pad is configured as a thin membrane with a partial slit and arranged such that the exposed portion of the filament (that which extends outside of the patient) passes through the slit. Hemostatic pads are typically comprised of a saturated gelatin sponge or felt-like material imbibed with one of three general categories of hemostatic agents, namely mechanical, active, and flowable (see e.g. Schreiber M A, Neveleff D J, Achieving hemostasis with topical hemostats: making clinically and economically appropriate decisions in the surgical and trauma settings. AORN Journal. 2011: 94, 5: S1-S20, doi: 10.1016/j.aorn.2011.09.018). Mechanical hemostatic agents (e.g. porcine gelatin, cellulose, bovine collagen, or polysaccharide spheres) activate the extrinsic coagulation cascade and form a matrix at the bleeding site. Active hemostatic agents (e.g., bovine thrombin, recombinant thrombin, or pooled human plasma thrombin) stimulate fibrinogen at the bleeding site to produce a fibrin clot. Flowable hemostatic agents are composed of either a porcine or bovine gelatin matrix plus thrombin. These flowable hemostats provide both a mechanical and an active hemostat in a single product. Unlike mechanical hemostatic agents, active and flowable hemostatic agents do not require the normal hemostatic pathway as part of their mechanism of action and therefore continue to function even in the presence of anticoagulants like heparin, which is most frequently a necessary medicinal therapy in interventional cardiology procedures.

Another aspect of the invention provides a hand-held control assembly which provides improved ease of use and control in the actuation of the delivery assembly to facilitate the deployment motions described above, i.e. the push cycle. The control assembly is comprised of a nested grasper handle and actuator that allow the control assembly to alternate between a default configuration and a deployed configuration with a single 90 degree twisting motion of the actuator followed by a single distal push of the actuator. The actuator component is affixed to the proximal margin of the pusher tube and is slide-ably nested in the grasper handle which is affixed to the proximal margin of the delivery shaft. When the actuator is fully displaced in the distal direction relative to the grasper handle, it motivates the pusher tube in the distal direction, thereby motivating the footplate to extrude out of the delivery shaft in a distal direction, and into the blood vessel, thus the push cycle.

Embodiments of the control assembly components may be comprised of a large variety of engineering thermoplastics such as ABS, Polycarbonate, Nylon (polyamide), HDPE, PEEK, Polypropylene, PVC, etc. These components can be fabricated by using a number of manufacturing techniques. Those include, but are not limited to, injection molding, machining, or additive manufacturing techniques such as 3-D printing, SLA, SLS, with effectiveness, as needed or desired.

The control assembly may also include a feature, or features, that provide the user with positive tactile and/or audible feedback indicating that complete actuation of the push cycle has occurred. One embodiment of such positive user feedback may be provided by a snap/detent feature on the grasper handle enclosure such that when the actuator is fully distally displaced, an audible/tactile ‘click’ is sensed by the operator, indicating that the actuator has reached its full stroke at the end of the push cycle and that the footplate has been fully displaced from within the delivery shaft and delivered inside the blood vessel lumen.

### BRIEF DESCRIPTION OF THE DRAWINGS

The present invention will be more fully understood and appreciated by reading the following Detailed Description in conjunction with the accompanying drawings, in which:

FIGS. 1a and 1b are perspective views of the footplate and filament in a pre-deployed a post-deployed orientation and configuration respectively, according to a preferred embodiment of the present invention.

FIG. 1c is a perspective view of the distal end of the filament, according to a preferred embodiment of the present invention.

FIG. 1d is a partially sectioned side view of the distal end of the filament and the connection method of the footplate thereto, according to a preferred embodiment of the present invention.

FIGS. 2a-2d are views depicting a method of piercing and swaging a footplate to facilitate fixation of a filament, according to an embodiment of the present invention.

FIG. 3a shows a partially sectioned side view of the introducer sheath, fully inserted in the patient's wrist and artery, according to an embodiment of the present invention.

FIG. 3b is a partially sectioned side view of the introducer sheath, pulled proximally such that its distal end is just slightly inside the patient's artery, according to an embodiment of the present invention.

FIG. 4 is a partially sectioned side view of the closure device partially inserted into the introducer sheath, according to an embodiment of the present invention.

FIG. 5a is a partially sectioned side view of the closure device fully inserted and locked onto the proximal hub of the introducer sheath, according to an embodiment of the present invention.

FIG. 5b is a partially sectioned perspective view of the closure device fully inserted and locked onto the proximal hub of the introducer sheath, according to an embodiment of the present invention.

FIG. 5c is a close-up detail of the distal end of the closure device in the default configuration after the closure device has been fully inserted and locked to the proximal hub of the introducer sheath, according to an embodiment of the present invention.

FIG. 6 is a partially sectioned perspective view of the closure device and introducer sheath in the configuration where the actuator has been turned 90 degrees clockwise, according to an embodiment of the present invention.

FIG. 7a is a partially sectioned side view of the closure device and introducer sheath where the actuator has been pushed in the distal direction to complete the push cycle, according to an embodiment of the present invention.

FIG. 7b is a partially sectioned perspective view of the closure device and introducer sheath (collectively, the closure system) of FIG. 7a, where the actuator has been pushed in the distal direction to complete the push cycle, according to an embodiment of the present invention.

FIG. 7c is a close-up, partially sectioned side view of the distal end of the closure device where the footplate has been delivered internal to the patient's artery, according to an embodiment of the present invention.

FIG. 8a is a partially sectioned side view of the closure device and introducer sheath in the configuration of a completed push cycle and where the closure device and the introducer sheath (together as a single unit) have been partially removed from the patient and where the footplate has been approximated against the wall of the artery, according to an embodiment of the present invention.

FIG. 8b is a close-up partially sectioned perspective view of FIG. 8a where the footplate is approximated against the anterior inside wall of the patient's artery and the filament extends proximally and is exposed proximal of the patient's skin, according to an embodiment of the present invention.

FIG. 9a is a partially sectioned side view of the closure device, the blood vessel and the tissue tract, and the filament is exposed and being grasped by the user in the configuration of the deployment sequence where the footplate is fully seated and the closure device deployment apparatus and the introducer sheath (together as a single unit) have been retracted proximally and upward away from the patient, according to an embodiment of the present invention.

FIG. 9b is a close-up of the distal portion of the closure device's implant (footplate and filament) shown in FIG. 9a, according to an embodiment of the present invention.

FIG. 10 is a partially sectioned side view of the closure device, the blood vessel and tissue tract, and the user posture in the configuration of the deployment sequence where the footplate is fully seated and the closure device deployment apparatus has been completely removed from the patient and the proximal margin of the filament is entirely exposed outside of the patient, according to an embodiment of the present invention.

FIGS. 11a-11d are partially sectioned side views of filament locking methods according to embodiments of the present invention.

FIG. 12 is a partially sectioned side view and a top view of a hemostatic pad positioned approximate to the patient's wrist for use in conjunction with a filament locking device (e.g. an alligator clip) according to an embodiment of the present invention.

FIG. 13 is a partially sectioned side view of the remaining portion of the closure device, the blood vessel and tissue tract, and the user posture in the configuration of the deployment sequence where the footplate has completely dissolved where all that remains of the closure device is the filament in the subcutaneous tissue tract and the temporary filament locking device (e.g. an alligator clip) which remains positioned at the skin surface, according to an embodiment of the present invention.

FIG. 14 is a partially sectioned side view of the remaining portion of the closure device, the blood vessel and tissue tract, and the user posture in the configuration after the footplate has completely dissolved and the operator has removed the temporary filament locking device (e.g. an alligator clip) and completely removes the filament from the patient by grasping and pulling the filament proximally, according to an embodiment of the present invention.

FIG. 15 is a sectioned side view of the blood vessel and tissue tract after the filament has been completely removed from the patient, rendering the arteriotomy fully hemostatic, according to an embodiment of the present invention.

### DETAILED DESCRIPTION

As mentioned earlier, vascular procedures are commonly performed through a puncture in either the radial artery or the ulnar artery. To close the puncture, often a compression device is utilized, which applies direct pressure to the skin surface on the inside of a patient's wrist, directed to compress the skin and subcutaneous tissue overlying the artery. These types of closure apparatus; however, can compress and collapse the arterial lumen, frequently rendering it non-patent. As an alternative to these ‘outside-in’ devices, the present invention describes a method and apparatus that creates hemostasis of an artery from the ‘inside-out’, i.e. a trans-radial or trans-ulnar closure device that applies expansive force rather than compressive force against the wall of a blood vessel.

The following detailed description contains certain references to positions identified as ‘distal’ and ‘proximal’. For clarity, these ‘distal’ and ‘proximal’ positions differ when referred to respective of; a) the closure device (the medical instrument), and; b) the patient.
a) When referring to the terms ‘distal’ and ‘proximal’ with respect to the closure device, distal is identified as the margin of the device that is forward of the user (closest to the patient), whereas proximal is identified as the margin of the device that is closer to the user, e.g. the control assembly is the most proximal portion of the closure device.
b) Quite oppositely, in cases where ‘distal’ and ‘proximal’ are referred to respective of positions on the patient, distal is identified as the position on the patient that is farther from the patient's heart, and proximal is identified as the position on the patient that is closer to the patient's heart, i.e. more cranial. By way of example, the tip of a patient's finger is more distal than the patient's wrist.

Reference will now be made in detail to the preferred embodiments of the present invention, examples of which are illustrated in the accompanying drawings.

In accordance with an embodiment of the present invention, the closure device 100, comprising a footplate 110 (the footplate may include any of the embodiments of the footplate, as discussed infra), and a filament 111 is provided and can be used to seal or close an opening formed through biological tissue, such as a percutaneously formed puncture (the puncture comprises the opening formed through the wall of the blood vessel and the tissue tract contiguous with the opening formed through the biological tissue, which extends through and to the skin overlying the blood vessel), an incision, or some other type of opening formed through biological tissue, such as a blood vessel, organ, or the like, to control (or prevent or stop) bleeding (or the flow of other biological fluid or tissue). For example, the closure device 100 of an embodiment of the present invention can be used to seal an arteriotomy 407, which is an opening, or incision, in an artery, such as the radial artery, and is formed in conjunction with a percutaneously formed puncture (an open tissue tract through the skin and tissue just above the blood vessel) by a clinician during a diagnostic or therapeutic intravascular surgical procedure.

In accordance with an embodiment of the present invention, and as elaborated in the subsequent descriptions, the closure device 100 may be in a pre-deployed configuration and position, or in a post-deployed configuration and position. A pre-deployed closure device configuration and position includes a configuration and position where the footplate 110 resides within the closure device 100. A post-deployed closure device configuration and position includes a configuration and position where the footplate 110 has been introduced through the arteriotomy 407 in the wall 123 of the blood vessel 121 and the footplate 110 resides inside the blood vessel 121 such that the footplate 110 is approximated against the blood vessel wall 123.

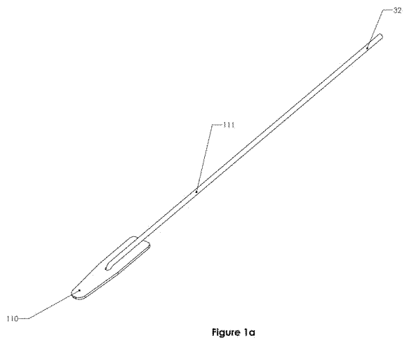

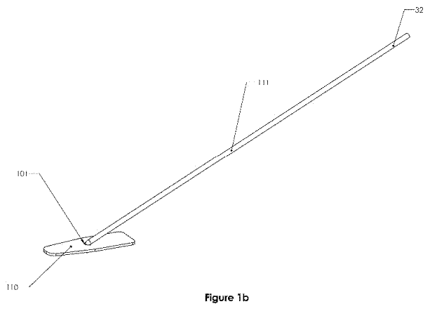

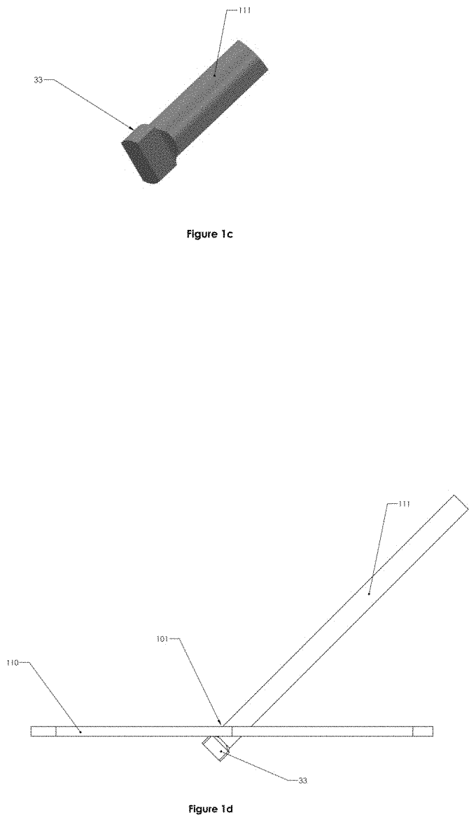

Referring now to the drawings in which like numbers refer to like parts throughout, FIG. 1a shows a footplate 110 according to an embodiment of the present invention. This embodiment shows a footplate 110 and a filament 111 in a pre-deployed closure device deployment configuration and position, wherein the footplate 110 is within the distal end of the delivery shaft 202 (not shown). The footplate 110 comprises a unitary flat or semi-flat plate with one thru-hole 101, through which a filament 111 passes and is tethered to the footplate 110 by means of a crimped section 33 (hidden) that is located at the most distal end of the filament 111 and is oriented at an angle substantially perpendicular to both the longitudinal axis of the footplate 110 and the longitudinal axis of the closure device 100, and whereby the proximal portion of the filament 111 is a single longitudinally-extending leg 32 which extends proximally, adjacent to the top surface of the footplate 110 and continues to extend proximally inside the pusher tube 201 (not shown) substantially parallel to the longitudinal axis of the footplate 110 and parallel to the longitudinal axis of the closure device 100 (not shown).

Turning to FIG. 1b, a footplate 110 according to an embodiment of the present invention is illustrated. This embodiment shows a footplate 110 and filament 111 of FIG. 1a in a post-deployed closure device deployment configuration and position, wherein the footplate 110 is displaced outside of the distal end of the delivery shaft 202 (not shown) and resides within the lumen of the blood vessel 121 (not shown) in an orientation substantially perpendicular to the longitudinal axis of the closure device 100 (not shown). The footplate 110 comprises a unitary flat or semi-flat plate with one thru-hole 101, through which a filament 111 passes and whereby the filament 111 is tethered to the footplate 110 by means of a crimped section 33 (hidden) that is located at the most distal end of the filament 111 and whereby the crimped section 33 (hidden) of the filament 111 is substantially perpendicular to the longitudinal axis of the footplate 110 and whereby the proximal portion of the filament 111 is a single longitudinally-extending leg 32 which is substantially perpendicular to the longitudinal axis of the footplate 110 and which continues to extend proximally inside the pusher tube 201 (not shown) and parallel to the longitudinal axis of the closure device 100 (not shown).

Turning to FIG. 1c, a close-up perspective view of the distal margin of the filament 111 described in FIGS. 1a and 1b, according to a preferred embodiment of the present invention is illustrated. This embodiment comprises a crimped, (permanently deformed) distal end 33 of the filament 111 which has been formed to be substantially larger than the diameter of the thru hole 101 (not shown) of the footplate 110 (not shown) such that the filament 111 remains tethered, or attached, to the footplate 110 until the footplate 110 has dissolved in the blood vessel 121 (not shown).

Turning to FIG. 1d, a side view of the connection method between the footplate 110 and the filament 111 shown in FIGS. 1a and 1b according to a preferred embodiment of the present invention is illustrated. This embodiment shows the footplate 110 with a filament 111 passing through the thru-hole 101 of the footplate 110 and extending in a substantially perpendicular orientation to the longitudinal axis of the footplate 110 such that the crimped distal end 33 of the filament 111 cannot pass through the through the thru-hole 101 of the footplate 110, thus maintaining the tethered connection between the footplate 110 and the filament 111 until such time that the footplate 110 has dissolved in the blood vessel 121 (not shown).

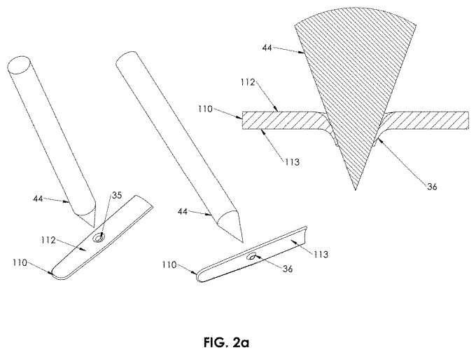

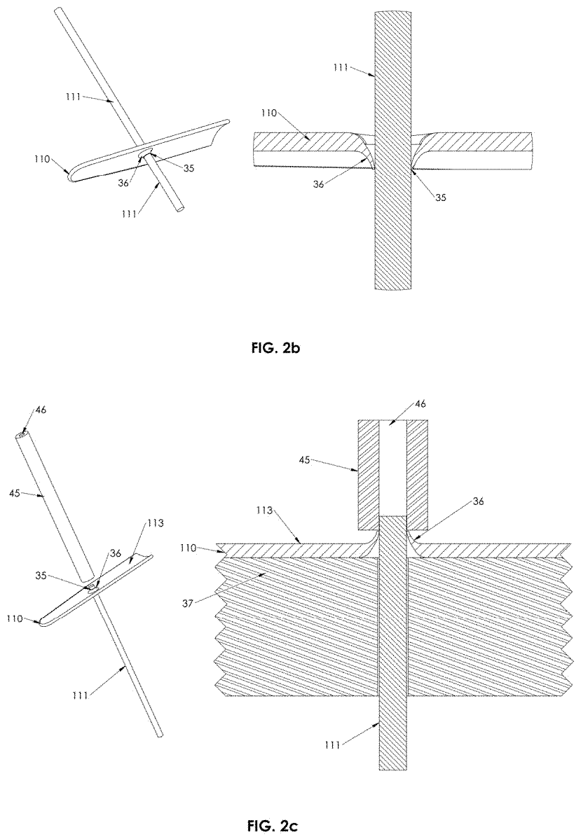

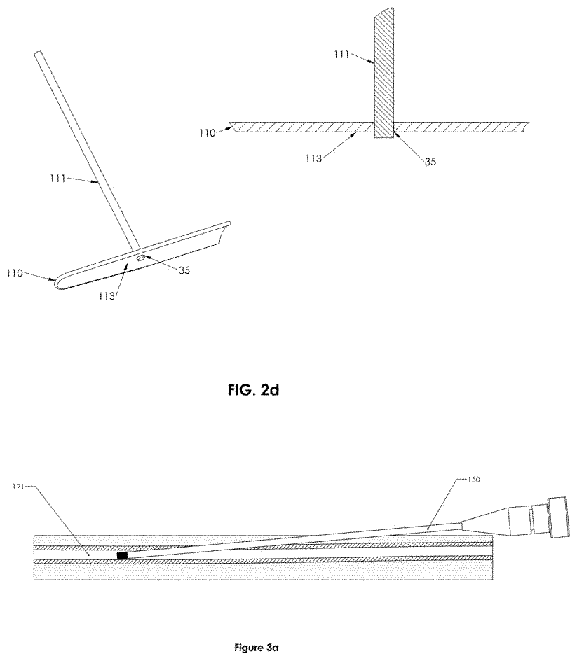

Turning to FIG. 2a, a footplate 110 according to an embodiment of the present invention is illustrated. This embodiment shows a footplate 110 whereby the footplate 110 comprises a unitary flat or semi-flat plate with a pierced thru-hole 35 whereby the pierced thru-hole 35 has been formed by a sharply tipped cylindrical piercing mandrel 44 that is introduced to the footplate 110 on the top surface of the footplate 112. The resulting geometry formed on (and through) the footplate 110 is that of a pierced thru-hole 35 surrounded by a dimpled, conically deformed feature 36, protruding from the bottom surface 113 of the footplate 110.

Turning to FIG. 2b, a footplate 110 and a filament 111 according to an embodiment of the present invention is illustrated. This embodiment shows the footplate of FIG. 2a with a filament 111 passing through the pierced thru-hole 35 in the conically deformed feature 36, and extending in a substantially perpendicular orientation to the longitudinal axis of the footplate 110.

Turning to FIG. 2c, a footplate 110 and a filament 111 according to an embodiment of the present invention is illustrated. This embodiment shows the footplate 110 of FIGS. 2a and 2b with a filament 111 whereby the filament 111 passes through the pierced thru-hole 35 in the footplate 110. A flat-ended reforming mandrel 45 is positioned such that filament 111 passes through a central hole 46 in the flat-ended mandrel 45 and the flat-ended mandrel 45 is positioned approximate to the conically deformed feature 36 on the bottom surface of the footplate 110 and perpendicular to the longitudinal axis of the footplate 110. Further, the footplate 110 is shown positioned against a supporting plate 37. This positioning (collectively, the pre-reforming configuration) readies the footplate 110 and filament 111 for the impending reforming of the dimpled, conically deformed 36 feature on the bottom surface 113 of the footplate 110 described infra.

Turning to FIG. 2d, a footplate 110 and a filament 111 according to an embodiment of the present invention is illustrated. This embodiment shows the footplate of FIGS. 2a, 2b and 2c with a filament 111 whereby the filament 111 is affixed to the footplate 110 by means of swaging, i.e. a local deformation force applied to the bottom surface 113 of the footplate 110 approximate to the dimpled, conically deformed feature 36 (not shown) around the thru-hole 35 on the footplate 110 in FIG. 2c. The force is applied at the region approximate to where the filament 111 passes, such that the conically deformed feature 36 (not shown) is reconfigured to become substantially flat, thus trapping and affixing the filament 111 to the footplate 110.

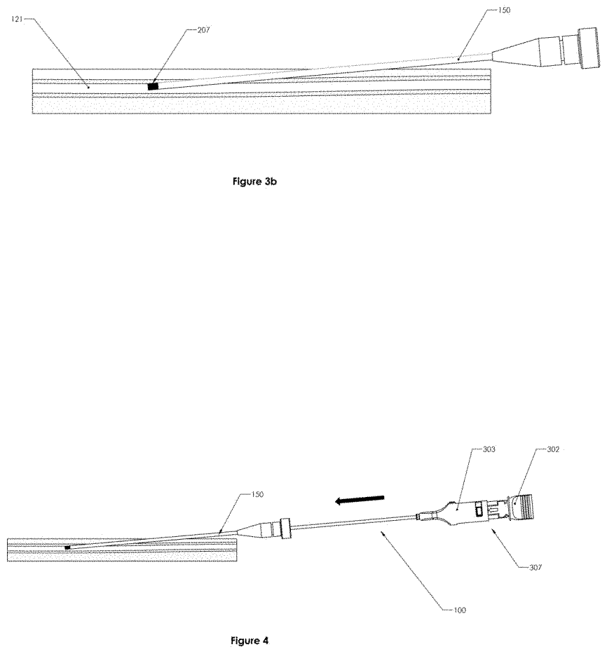

Turning to FIG. 3a, an introducer sheath 150 is illustrated according to an embodiment of the present invention. This embodiment shows a cut-away (partially exposed) left side of the introducer sheath 150 fully introduced into a blood vessel 121 at the end of the interventional or diagnostic procedure.

Turning to FIG. 3b, the introducer sheath 150 of FIG. 3a is shown after it has been pulled proximally such that its distal tip resides only slightly inside of the blood vessel 121, according to an embodiment of the present invention. Also illustrated, according to an embodiment of the present invention, is a radiopaque marker band 207 on the distal portion of the introducer sheath such that the distal portion of the introducer sheath 150 is positioned in the blood vessel 121 such that the radiopaque marker band 207 remains fully inside the blood vessel 121 and can be identified as such under fluoroscopic visualization, as necessary. Such a pre-existing radiopaque marker band 207 is available as a standard feature in some commercially available introducer sheaths.

Turning to FIG. 4, a left side view of the closure device 100 is shown, according to an embodiment of the present invention. This embodiment shows the left side of the closure device 100 including its proximal control assembly 307 (i.e. the grasper 303 and the actuator 302) in the configuration where the closure device 100 has been partially inserted into the introducer sheath 150.

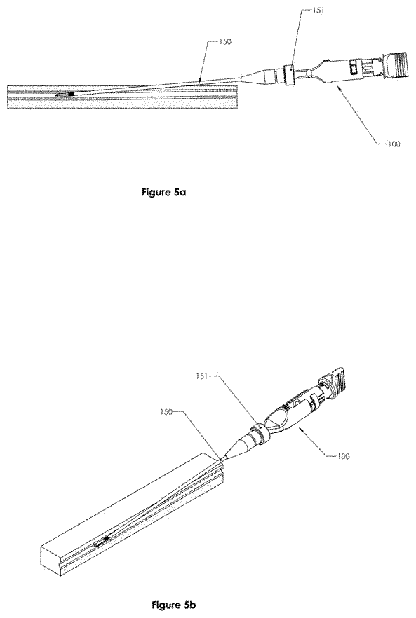

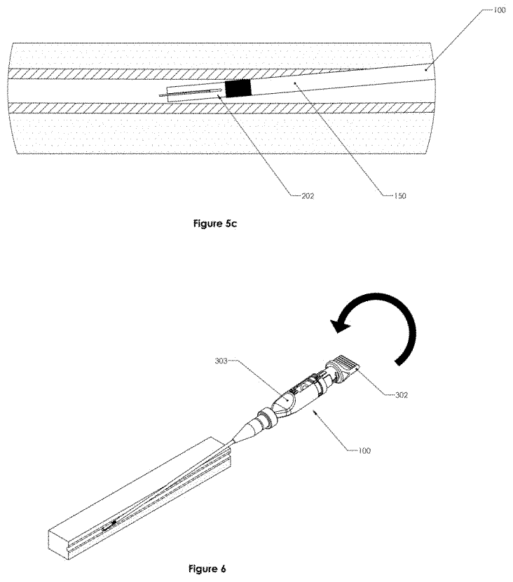

Turning to FIG. 5a, a left side view of the closure device 100 according to an embodiment of the present invention is illustrated. This embodiment shows the closure device 100 fully inserted into the introducer sheath 150 and locked to the proximal hub 151 of the introducer sheath 150.

Turning to FIG. 5b, is a perspective view of the closure device 100 fully inserted and locked to the proximal hub 151 of the introducer sheath 150 shown in FIG. 5a, according to an embodiment of the present invention is illustrated.

Turning to FIG. 5c, a close-up view of the distal portion of the closure device 100 shown in FIG. 5a, in accordance with an embodiment of the present invention is illustrated. This embodiment shows the delivery shaft 202 extending distally beyond the distal end of the introducer sheath 150, i.e. after the closure device 100 has been fully inserted and locked to the proximal hub 151 (not shown) of the introducer sheath 150.

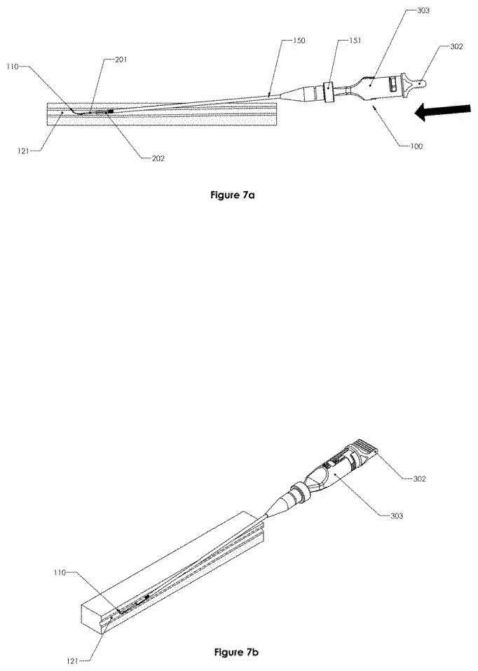

Turning to FIG. 6, the closure device 100 according to an embodiment of the present invention is illustrated. This embodiment shows a perspective view of the closure device 100 after the actuator 302 has been rotated 90 degrees with respect to the grasper handle 303, but prior to the actuator 302 being pushed in the distal direction to complete the push cycle (as is shown infra).

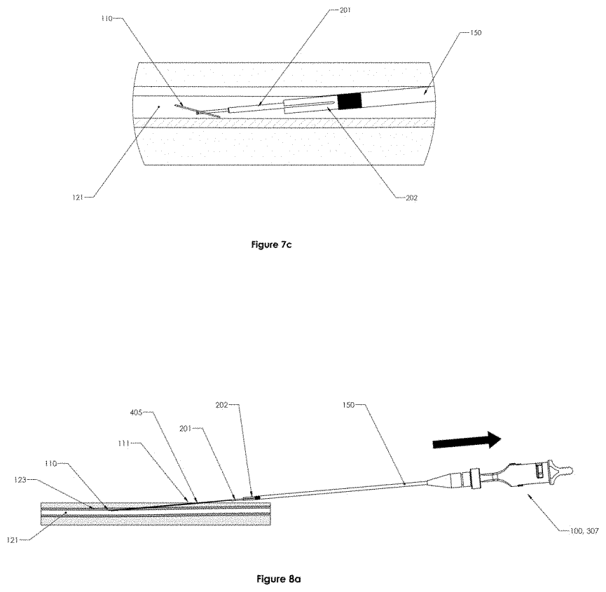

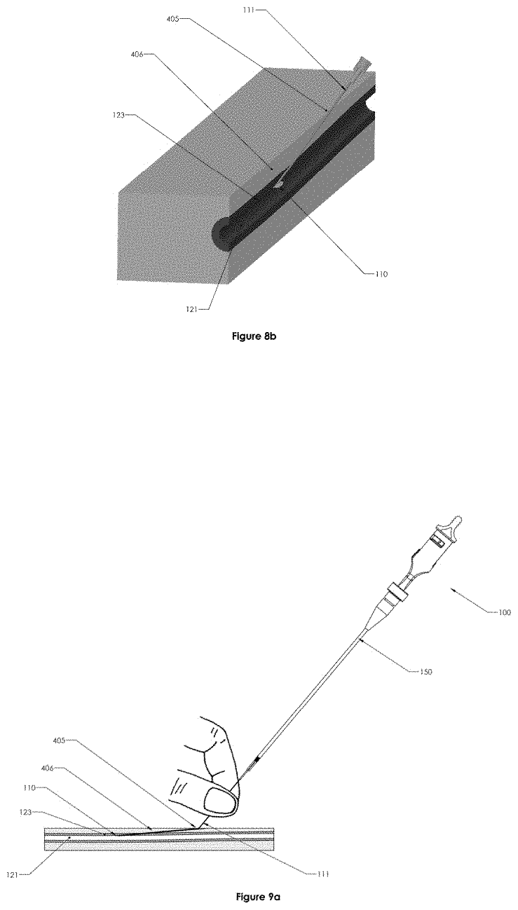

Turning to FIG. 7a, a partially sectioned left side view of the closure device 100 according to an embodiment of the present invention is illustrated. This embodiment shows closure device 100 (including its proximal control assembly) locked to the proximal hub 151 of the introducer sheath 150, after the actuator 302 has been pushed distally relative to the grasper handle 303 such that the footplate 110 has been motivated distally by the pusher tube 201 and released from within the delivery shaft 202 and into the blood vessel 121 that requires sealing, i.e. the completed push cycle deployment configuration and position.

Turning to FIG. 7b, a partially sectioned perspective view of FIG. 7a is shown, in accordance with an embodiment of the present invention. This embodiment also depicts the completed distal movement of the actuator 302 relative to the grasper handle 303, i.e. the completed push cycle, and shows the displaced footplate 110 in the blood vessel 121.

Turning to FIG. 7c, is a close-up view of the distal portion of the introducer sheath 150 and closure device 100 (as a mated assembly) shown in FIG. 7a, in accordance with an embodiment of the present invention. This embodiment shows the pusher tube 201 after it has been motivated distally, thus displacing the footplate 110 out of the delivery shaft 202 and into the lumen of the blood vessel 121.

Turning to FIG. 8a, a partially sectioned left side view of the closure device 100 according to an embodiment of the present invention is illustrated. The embodiment shows the closure device 100, including its proximal control assembly 307, the footplate 110, the filament 111, the pusher tube 201, and delivery shaft 202 in a post-push cycle closure device deployment configuration and position where the entire assembly (the closure device 100 and the introducer sheath 150, together as a single unit) has been partially retracted out of the patient in the proximal direction such that the filament 111 is exposed at a position proximal of the skin incision 405. This proximal retraction of the entire system then seats the footplate 110 to be approximated against the inside wall 123 of the blood vessel 121 to affect immediate hemostasis.

Turning to FIG. 8b, a close-up partially sectioned perspective view of the distal position of the closure device 100 shown in FIG. 8a is illustrated, according to an embodiment of the present invention. This embodiment shows the footplate 110 tethered to the filament 111 and positioned approximate to the inside wall 123 of the blood vessel 121 and longitudinally aligned with the longitudinal axis of the blood vessel 121. Also shown is the filament 111 extending proximally from the footplate 110 through the vessel wall 123 and the subcutaneous tissue 406 and further proximally where the filament 111 is exposed proximal (outside) of the skin incision 405.

Turning to FIG. 9a, a partially sectioned left side view of the closure device 100, according to an embodiment of the present invention is illustrated. The embodiment shows the closure device 100 in a closure device deployment configuration and position where the closure device 100 and the introducer sheath 150 (together, as a mated assembly) has been retracted proximally and upward away from the patient (fully outside of the percutaneous tissue tract in the subcutaneous tissue 406 and the skin incision 405) and positioned to expose enough of the filament 111 to allow the user to grasp the filament 111 and apply gentle tension such that the footplate 110 is held in close approximation with the vessel wall 123 to maintain hemostasis for a period of time before a temporary filament locking device may be applied as described infra.

Turning to FIG. 9b, a close-up view of the distal portion of the closure device 100 shown in FIG. 9a according to an embodiment of the present invention is illustrated. The embodiment shows the blood vessel 121, the percutaneous tissue tract in the subcutaneous tissue 406, and the skin incision 405 (not shown) in a closure device deployment configuration and position where the footplate 110 is fully seated (approximated) against the vessel wall 123.

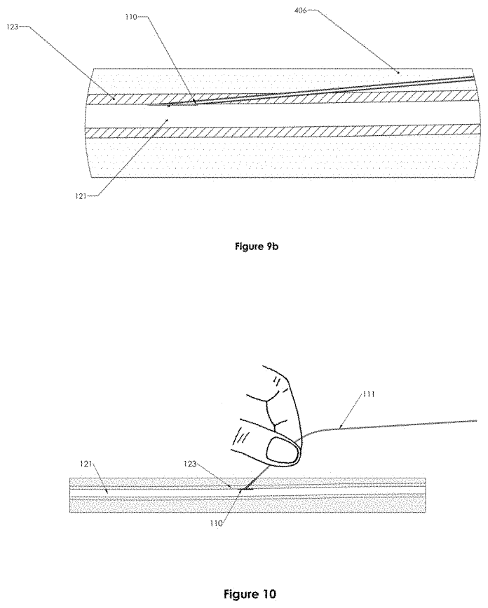

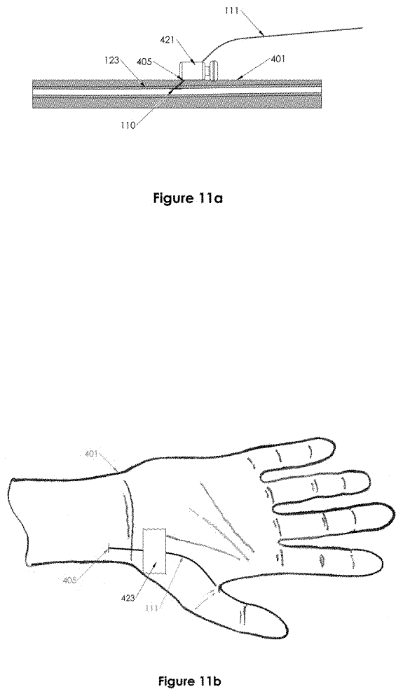

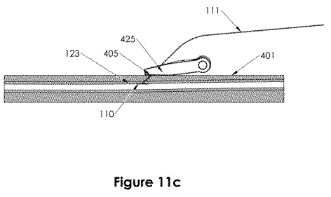

Turning to FIG. 10, a partially sectioned left side view of the blood vessel 121 with the footplate 110 fully approximated and the user hand posture according to an embodiment of the present invention is illustrated. The embodiment shows the closure device 100 (not shown) completely removed from the filament 111 (and the operative site) and the filament 111 being grasped by the operator in order to maintain gentle tension on the filament 111 to facilitate continued contact of the footplate 110 against the vessel wall 123, until a means of temporary securement can be applied to the filament 111 (as is shown infra).

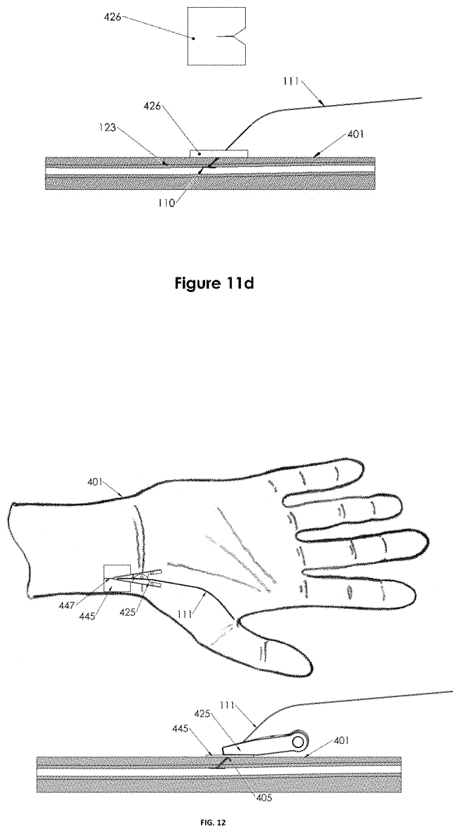

Turning to FIG. 11a, a temporary filament locking device is illustrated, according to an embodiment of the present invention. In a partially sectioned left side view, the embodiment illustrates a cord lock 421 holding tension on the filament 111 approximate to the skin incision 405 on the patient's wrist 401 such that the tension on the filament 111 maintains approximation of the footplate 110 with the vessel wall 123 for a period of time such that the footplate 110 has substantially dissolved.

Turning to FIG. 11b shows a temporary filament locking means according to an embodiment of the present invention. In a top view, the embodiment illustrates an adhesive tape filament locking means 423 holding tension on the filament 111 approximate to the skin surface on the patient's wrist 401 and distal (on the patient) of the skin incision 405, or further distal on the patient (e.g. on the palm of the patient's hand), such that the tension on filament 111 maintains approximation of the footplate 110 (not shown) with the vessel wall 123 (not shown) for a period of time such that the footplate 110 (not shown) has substantially dissolved.

Turning to FIG. 11c shows a temporary filament locking device according to an embodiment of the present invention. In a partially sectioned left side view, the embodiment illustrates an alligator clip 425 holding tension on the filament 111 approximate to the skin incision 405 on the patient's wrist 401 such that the tension on the filament 111 maintains approximation of the footplate 110 with the vessel wall 123 for a period of time such that the footplate 110 has substantially dissolved.

Turning to FIG. 11d shows a temporary filament locking device according to an embodiment of the present invention. In a partially sectioned left side view, the embodiment illustrates a partially slit closed cell polymer foam pad 426 holding tension on the filament 111 approximate to the skin incision 405 on the patient's wrist 401 such that the tension on the filament 111 maintains approximation of the footplate 110 with the vessel wall 123 for a period of time such that the footplate 110 has substantially dissolved.

Turning to FIG. 12 shows a hemostatic pad 445 according to an embodiment of the present invention. In both a partially sectioned left side view and a top view showing the patient's hand and wrist 401, the hemostatic pad 445 is positioned such that it is in contact with the skin incision 405 on the patient's wrist 401, and further arranged such that the exposed portion of the filament 111 passes through a partial slit 447 in the hemostatic pad 445. The embodiment further illustrates the hemostatic pad 445 being sandwiched between a filament locking device (e.g. an alligator clip 425) and the skin incision 405 such that the hemostatic pad 445 is captured in firm contact against the skin of the patient's wrist 401.

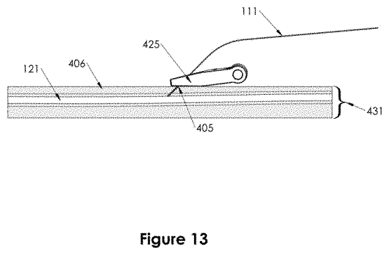

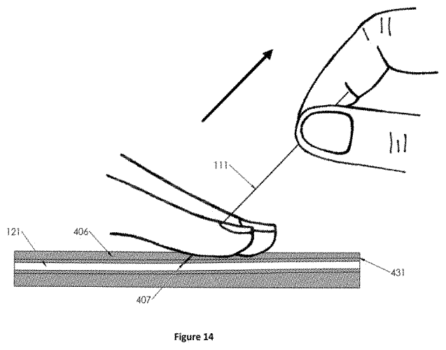

Turning to FIG. 13, a partially sectioned left side view of the blood vessel 121, the percutaneous tissue tract in the subcutaneous tissue 406, the skin incision 405, the filament 111, and the temporary filament locking device (e.g. an alligator clip 425) according to an embodiment of the present invention is illustrated. The embodiment shows the tissue layers 431 in a condition after a period of time has elapsed such that the footplate 110 (not shown) has fully dissolved in the blood vessel 121, i.e. the only remaining portion of the closure device 100 (not shown) is the filament 111 (in the percutaneous tissue tract in the subcutaneous tissue 406 and extending out of the skin incision 405) and the temporary filament locking device (e.g. an alligator clip 425), both of which will be removed from the patient (as is shown and described infra).

Turning to FIG. 14, a partially sectioned left side view of the blood vessel 121, the percutaneous tissue tract in the subcutaneous tissue 406, the skin incision 405 (hidden), the filament 111, and the user posture according to an embodiment of the present invention is illustrated. The embodiment shows the tissue layers 431 in a condition after a period of time has elapsed such that the footplate 110 (not shown) has substantially dissolved, or absorbed, and the temporary filament locking device (e.g. an alligator clip 425, not shown) having been removed from the filament 111. The filament 111 is grasped by the operator and pulled in a proximal direction (away from the patient to completely remove the filament 111 from the patient) while simultaneously holding pressure on the skin surface of the patient's wrist 401 such that the operator's forefinger and middle finger straddle the filament thus providing equal and opposite downward contact force on the skin and subcutaneous tissue 406 (i.e. providing support and resistance to upward tissue displacement) while tension is applied to the filament during removal. At this juncture, the filament 111 is completely removed from the patient and there exists a fully hemostatic condition (i.e. complete cessation of bleeding) at the arteriotomy 407.

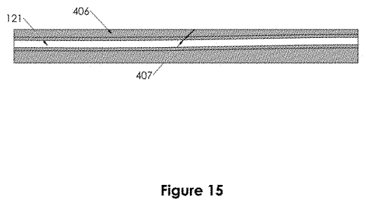

Turning to FIG. 15, a sectioned left side view of the blood vessel 121 and tissue tract in the subcutaneous tissue 406 after the filament 111 (not shown) has been completely removed from the patient, rendering the arteriotomy 407 in the blood vessel 121 fully hemostatic, according to an embodiment of the present invention.

It should be understood that the values used above are only representative values, and other values may be in keeping with the spirit and intention of this disclosure.

While several inventive embodiments have been described and illustrated herein with reference to certain exemplary embodiments, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein (and it will be understood by one skilled in the art that various changes in detail may be effected therein without departing from the spirit and scope of the invention as defined by claims that can be supported by the written description and drawings). More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Further, where exemplary embodiments are described with reference to a certain number of elements it will be understood that the exemplary embodiments can be practiced utilizing either less than or more than the certain number of elements.

All references, including publications, patent applications, and patents, cited herein are hereby incorporated by reference to the same extent as if each reference were individually and specifically indicated to be incorporated by reference and were set forth in its entirety herein.

All definitions, as defined and used herein, should be understood to control over dictionary definitions, definitions in documents incorporated by reference, and/or ordinary meanings of the defined terms.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if not directly attached to where there is something intervening.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A tissue puncture closure assembly, comprising:
a closure device, comprising:
a footplate composed of a biodegradable metal with an opening extending therethrough;
a filament passing through the opening, the filament having a first end and a second end; and
wherein the second end of the filament has a diameter that is larger than a diameter of the opening;

2. The assembly of claim 1, wherein the biodegradable metal is magnesium alloy.

3. The assembly of claim 1, further comprising a surface modification layer on the footplate which affects the rate of dissolution of the footplate.

4. The assembly of claim 3, wherein the surface modification layer is composed of an acid and an accelerant.

5. The assembly of claim 4, wherein the acid is an inorganic acid selected from a group consisting of sulfuric acid, nitric acid, hydrochloric acid, phosphoric acid, and phosphonic acid.

6. The assembly of claim 4, wherein the acid is an organic acid selected from a group consisting of citric acid, tartaric acid, acetic acid, and oxalic acid.

7. The assembly of claim 4, wherein the accelerant is a soluble transition metal salt.

8. The assembly of claim 1, wherein the filament is composed of a bioabsorbable material selected from a group consisting of Polyglycolic acid polymer, Polyglactin copolymer, Poliglecaprone copolymer, Polydioxanone polymer, and Catgut.

9. The assembly of claim 1, wherein the filament is composed of a non-bioabsorbable material selected from a group consisting of Polypropylene, Nylon (polyamide), Polyester, PVDF, PTFE, ePTFE, silk, stainless steel, and nitinol.

10. The assembly of claim 1, wherein the filament is either a braided construction or a monofilament construction.

11. The assembly of claim 1, further comprising:
a delivery device comprising a cannulated delivery shaft and a cannulated pusher tube, the cannulated pusher tube concentrically positioned within the cannulated delivery shaft;
wherein the footplate is within a distal end of the delivery shaft.

12. The assembly of claim 11, wherein the filament extends proximally through the pusher tube.

13. The assembly of claim 11, wherein at least one of the cannulated delivery shaft and the cannulated pusher tube is composed of a polymer selected from a group consisting of Pebax, Silicone, Nylon (polyamide), Polyurethane, PTFE, FEP, ETFE, and HDPE.

14. The assembly of claim 11, wherein distal force on the cannulated pusher tube relative to the cannulated delivery shaft moves the footplate distally through the delivery shaft.

15. The assembly of claim 11, further comprising one or more axial slits extending at least partially through the cannulated delivery shaft.
